SEQUENCE LISTING (6 sequences)

SEQ ID NO: 1 - DNA, 43 bp, Homo sapiens
caccgaattctgtgtccctactatggtcagaaagcctgttgtg

SEQ ID NO: 2 - DNA, 50 bp, Homo sapiens
taacttatcttcttctggtacaacttccagtattatttgtaatgttctgg

SEQ ID NO: 3 - DNA, 1528 bp, Homo sapiens
caccgaattctgtgtccctactatggtcagaaagcctgttgtgtccaccatctccaaaggaggttacctgcagggaaatgttaacgggaggctgccttccctgggcaacaaggagccacctgggcaggagaaagtgcagctgaagaggaaagtcactttactgaggggagtctccattatcattggcaccatcattggagcaggaatcttcatctctcctaagggcgtgctccagaacacgggcagcgtgggcatgtctctgaccatctggacggtgtgtggggtcctgtcactatttggagctttgtcttatgctgaattgggaacaactataaagaaatctggaggtcattacacatatattttggaagtctttggtccattaccagcttttgtacgagtctgggtggaactcctcataatacgccctgcagctactgctgtgatatccctggcatttggacgctacattctggaaccattttttattcaatgtgaaatccctgaacttgcgatcaagctcattacagctgtgggcataactgtagtgatggtcctaaatagcatgagtgtcagctggagcgcccggatccagattttcttaaccttttgcaagctcacagcaattctgataattatagtccctggagttatgcagctaattaaaggtcaaacgcagaactttaaagacgccttttcaggaagagattcaagtattacgcggttgccactggctttttattatggaatgtatgcatatgctggctggttttacctcaactttgttactgaagaagtagaaaaccctgaaaaaaccattccccttgcaatatgtatatccatggccattgtcaccattggctatgtgctgacaaatgtggcctactttacgaccattaatgctgaggagctgctgctttcaaatgcagtggcagtgaccttttctgagcggctactgggaaatttctcattagcagttccgatctttgttgccctctcctgctttggctccatgaacggtggtgtgtttgctgtctccaggttattctatgttgcgtctcgagagggtcaccttccagaaatcctctccatgattcatgtccgcaagcacactcctctaccagctgttattgttttgcaccctttgacaatgataatgctcttctctggagacctcgacagtcttttgaatttcctcagttttgccaggtggctttttattgggctggcagttgctgggctgatttatcttcgatacaaatgcccagatatgcatcgtcctttcaaggtgccactgttcatcccagctttgttttccttcacatgcctcttcatggttgccctttccctctattcggacccatttagtacagggattggcttcgtcatcactctgactggagtccctgcgtattatctctttattatatgggacaagaaacccaggtggtttagaataatgtcggagaaaataaccagaacattacaaataatactggaagttgtaccagaagaagataagttatga

SEQ ID NO: 4 - protein, 501 aa, Homo sapiens
MVRKPVVSTISKGGYLQGNVNGRLPSLGNKEPPGQEKVQLKRKVTLLRGVSIIIGTIIGAGIFISPKGVLQNTGSVGMSLTIWTVCGVLSLFGALSYAELGTTIKKSGGHYTYILEVFGPLPAFVRVWVELLIIRPAATAVISLAFGRYILEPFFIQCEIPELAIKLITAVGITVVMVLNSMSVSWSARIQIFLTFCKLTAILIIIVPGVMQLIKGQTQNFKDAFSGRDSSITRLPLAFYYGMYAYAGWFYLNFVTEEVENPEKTIPLAICISMAIVTIGYVLTNVAYFTTINAEELLLSNAVAVTFSERLLGNFSLAVPIFVALSCFGSMNGGVFAVSRLFYVASREGHLPEILSMIHVRKHTPLPAVIVLHPLTMIMLFSGDLDSLLNFLSFARWLFIGLAVAGLIYLRYKCPDMHRPFKVPLFIPALFSFTCLFMVALSLYSDPFSTGIGFVITLTGVPAYYLFIIWDKKPRWFRIMSEKITRTLQIILEVVPEEDKL

SEQ ID NO: 5 - DNA, 1409 bp, Homo sapiens
caccgaattctgtgtccctactatggtcagaaagcctgttgtgtccaccatctccaaaggaggttacctgcagggaaatgttaacgggaggctgccttccctgggcaacaaggagccacctgggcaggagaaagtgcagctgaagaggaaagtcactttactgaggggagtctccattatcattggcaccatcattggagcaggaatcttcatctctcctaagggcgtgctccagaacacgggcagcgtgggcatgtctctgaccatctggacggtgtgtggggtcctgtcactatttggagctttgtcttatgctgaattgggaacaactataaagaaatctggaggtcattacacatatattttggaagtctttggtccattaccagcttttgtacgagtctgggtggaactcctcataatacgccctgcagctactgctgtgatatccctggcatttggacgctacattctggaaccattttttattcaatgtgaaatccctgaacttgcgatcaagctcattacagctgtgggcataactgtagtgatggtcctaaatagcatgagtgtcagctggagcgcccggatccagattttcttaaccttttgcaagctcacagcaattctgataattatagtccctggagttatgcagctaattaaaggtcaaacgcagaactttaaagacgccttttcaggaagagattcaagtattacgcggttgccactggctttttattatggaatgtatgcatatgctggctggttttacctcaactttgttactgaagaagtagaaaaccctgaaaaaaccattccccttgcaatatgtatatccatggccattgtcaccattggctatgtgctgacaaatgtggcctactttacgaccattaatgctgaggagctgctgctttcaaatgcagtggcagtgaccttttctgagcggctactgggaaatttctcattagcagttccgatctttgttgccccctcctctaccagctgttattgttttgcaccctttgacaatgataatgctcttctctggagacctcgacagtcttttgaatttcctcagttttgccaggtggctttttattgggctggcagttgctgggctgatttatcttcgatacaaatgcccagatatgcatcgtcctttcaaggtgccactgttcatcccagctttgttttccttcacatgcctcttcatggttgccctttccctctattcggacccatttagtacagggattggcttcgtcatcactctgactggagtccctgcgtattatctctttattatatgggacaagaaacccaggtggtttagaataatgtcggagaaaataaccagaacattacaaataatactggaagttgtaccagaagaagataagtta

SEQ ID NO: 6 - protein, 409 aa, Homo sapiens
MVRKPVVSTISKGGYLQGNVNGRLPSLGNKEPPGQEKVQLKRKVTLLRGVSIIIGTIIGAGIFISPKGVLQNTGSVGMSLTIWTVCGVLSLFGALSYAELGTTIKKSGGHYTYILEVFGPLPAFVRVWVELLIIRPAATAVISLAFGRYILEPFFIQCEIPELAIKLITAVGITVVMVLNSMSVSWSARIQIFLTFCKLTAILIIIVPGVMQLIKGQTQNFKDAFSGRDSSITRLPLAFYYGMYAYAGWFYLNFVTEEVENPEKTIPLAICISMAIVTIGYVLTNVAYFTTINAEELLLSNAVAVTFSERLLGNFSLAVPIFVAPSSTSCYCFAPFDNDNALLWRPRQSFEFPQFCQVAFYWAGSCWADLSSIQMPRYASSFQGATVHPSFVFLHMPLHGCPFPLFGPI

(19) United States
(12) Patent Application Publication
(10) Pub. No.: US 2003/0224454 A1
(43) Pub. Date: Dec. 4, 2003

(54) Human solute carrier family 7, member 11 (hSLC7A11)

(76) Inventors: Rolf Peter Ryseck, Ewing, NJ (US); Matthew V. Lorenzi, Philadelphia, PA (US); David K. Bol, Gaithersburg, MD (US)

Correspondence Address: STEPHEN B. DAVIS, BRISTOL-MYERS SQUIBB COMPANY, PATENT DEPARTMENT, P O BOX 4000, PRINCETON, NJ, 08543-4000, US

(21) Appl. No.: 10/447,920

(22) Filed: May 29, 2003

Related U.S. Application Data
(60) Provisional application No. 60/384,306, filed on May 30, 2002.

(51) Int. Cl. G01N 33/53; C07H 21/04; C07K 14/705; C12P 21/02; C12N 5/06

(52) U.S. Cl. 435/7.1; 435/69.1; 435/320.1; 435/325; 530/350; 536/23.5

(57) ABSTRACT

Human solute carrier family 7, member 11 (hSLC7A11) polynucleotides and polypeptides. Also provided are expression vectors, recombinant host cells and processes for producing recombinant host cells, processes for producing said polypeptides, and methods for identifying receptors that are capable of binding to a solute carrier family 7, member 11 molecule.

This application claims the benefit of U.S. Provisional Application No. 60/384,306 filed May 30, 2002, whose contents are incorporated by reference in its entirety.

### BACKGROUND OF THE INVENTION

The transport of amino acids across cellular membranes is adapted to the needs of specific cells as well as to local and systemic requirements. For instance, active amino acid uptake is a necessity for growing cells. Various members of the novel family of glycoprotein-associated amino acid transporters or solute carrier family 7 (SLC7) have been identified and shown to play roles in cellular uptake and/or basolateral extrusion of basic and neutral amino acids (Rossier et al., J. Biol. Chem 274: 34948-34954 (1999)). These permease-related proteins with twelve transmembrane domains require heterodimerization with a type II heavy chain glycoprotein, such as 4F2 heavy chain (4F2hc) or rBAT to express their function. The association of glycoprotein-associated amino acid transporters with 4F2hc or possibly rBAT is a prerequisite for the transporters to reach the cell surface (Mastroberardino et al., Nature 395:288-291 (1998)). In epithelial tissues, for example, trafficking of the 4F2hc subunit ensures a basolateral location, where the transporters allow the release of neutral or cationic amino acids into the blood. (Broer et al., Biochem. J. 349:787-795 (2000); Verrey et al., J. Membr. Biol. 172:181-192 (1999); Christensen, H., Physiol Rev. 70:43-77 (1990); and Broer, Nova Acta Leopoldinana 306:79-91 (1998)).

Members of the SLC7 family of transporters are evolutionarily conserved. Possible involvement of SLC7A5 (LAT1) in colon cancer has been reported (Wolf et al., Cancer Res. 56:5012-5022 (1996)). SLC7A7 has been implicated in lysinuric protein intolerance (LPI) (Torrents et al., Nature Genet. 21:293-296 (1999); Borsani et al., Nature Genet. 21:297-301 (1999)). Other members of this family (SLC7A9 and SLC7A10) have been implicated in cystinurea (Feliubadalo et al., Nature Genet. 23:52-57 (1999); Leclerc et al., Mol. Genet. Metab. 73:333-339 (2001).

Thus, the identification of unknown amino acid transporters that play an essential role in the existence and maintenance of cells, tissues, organs and the living body has the potential to clarify the causes or onset of diseases associated with transporter function. In addition, the identification of an amino acid transporter that is specifically expressed in abnormal cells directly participating in the given symptoms, such as cancer cells, and plays a role of supplying an amino acid to the abnormal cells can aid in the development of therapeutic methods of treatment of said symptoms.

Therefore, the development of therapeutics that modulate members of the SLC7 family (i.e., act as antagonists or agonists of SLC7 members) is important to treat diseases related to cellular uptake and/or basolateral extrusion of amino acids, such as cancer.

### SUMMARY OF THE INVENTION

The present invention provides human solute carrier family 7, member 11 (hSLC7A10) polynucleotides and polypeptides that have homology to other solute carrier family 7 members (SLC7s).

In one aspect, the invention provides isolated polynucleotides comprising: (a) a nucleotide sequence encoding a solute carrier family 7, member 11 polypeptide wherein the amino acid sequence of the polypeptide and the amino acid sequence of at least one of SEQ ID NO: 4 and SEQ ID NO: 6 have at least 80% sequence identity; or (b) the complement of the nucleotide sequence, wherein the complement and the nucleotide sequence contain the same number of nucleotides and are 100% complementary. In another aspect, the sequence identity is at least 85%, at least 90%, at least 95%, at least 96%, at least 97%, at least 98%, or at least 99%. In another aspect, the isolated polynucleotides of the invention encode the polypeptide of SEQ ID NO: 4 or SEQ ID NO: 6. In yet a further aspect of the invention, the isolated polynucleotides comprise SEQ ID NO: 3 or SEQ ID NO: 5.

The invention also provides expression vectors that comprise a polynucleotide of the invention and an expression control sequence operatively linked to the polynucleotide.

The invention further provides processes for producing a recombinant host cell comprising transforming or transfecting a host cell with an expression vector of the invention such that the host cell, under appropriate culture conditions, produces a solute carrier family 7, member 11 polypeptide. The invention also includes recombinant host cells produced by this process.

The invention further includes isolated solute carrier family 7, member 11 polypeptides comprising an amino acid sequence that has at least 80% sequence identity to at least one of the amino acid sequences of SEQ ID NO: 4 or SEQ ID NO: 6. In another aspect, the sequence identity is at least 85%, at least 90%, at least 95%, at least 96%, at least 97%, at least 98%, or at least 99%. In yet another aspect, the isolated solute carrier family 7, member 11 polypeptides comprise the amino acid sequence of SEQ ID NO: 4 or SEQ ID NO: 6.

The invention also includes processes for producing a solute carrier family 7, member 11 polypeptide comprising culturing a recombinant host cell of the invention under conditions sufficient for the production of said polypeptide and recovering the polypeptide from the culture.

The invention also provides methods for identifying a receptor which is capable of binding to a solute carrier family 7, member 11 molecule or a fragment thereof, said method comprising the steps of: (a) reacting a solute carrier family 7, member 11 polypeptide of the invention or a fragment thereof with a candidate receptor under conditions which permit the formation of receptor-solute carrier family 7, member 11 polypeptide complexes; and (b) assaying for candidate receptor-solute carrier family 7, member 11 polypeptide complexes or for activation of the candidate receptor, wherein the presence of at least one of candidate receptor-solute carrier family 7, member 11 polypeptide complexes and activation of the candidate receptor indicates that the candidate receptor is capable of binding to said solute carrier family 7, member 11 molecule or said fragment thereof.

### BRIEF DESCRIPTION OF THE FIGURES

FIGS. 1A-1E show the polynucleotide sequence of full length hSLC7A11 (SEQ ID NO: 3) aligned with the sequence for the hSLC7A11 splice variant (SEQ ID NO: 5).

FIGS. 2A-B show the amino acid sequence of full length hSLC7A11 (SEQ ID NO: 4) aligned with the sequence for the hSLC7A11 splice variant (SEQ ID NO: 6).

FIGS. 3A-F show the alignment of the amino acid sequence for full length hSLC7A11 (SEQ ID NO: 4) and hSLC7A11 splice variant (SEQ ID NO: 6) with other members of the SLC7 family.

### DETAILED DESCRIPTION OF THE INVENTION

The invention includes a human amino acid transporter of the SLC7 family and a splice variant of said transporter, hereinafter collectively referred to as “hSLC7A11.” The polynucleotide and polypeptide sequences of the invention have homology to other solute carrier family 7 members (SLC7s).

The hSLC7A11 polypeptides of the invention can be produced by: (1) inserting the cDNA of the disclosed hSLC7A11 into an appropriate expression vector; (2) transfecting the expression vector into an appropriate transfection host(s); (3) growing the transfected host(s) in appropriate culture media; and (4) purifying the receptor protein from the culture media.

The invention therefore provides a purified and isolated nucleic acid molecule, preferably a DNA molecule, having a sequence that encodes for a hSLC7A11, or an oligonucleotide fragment of the nucleic acid molecule which is unique to the hSLC7A11 of the invention. In a preferred embodiment of the invention, the purified and isolated nucleic acid molecule has the sequence as shown in SEQ ID NO: 3 or SEQ ID NO: 5.

The invention also contemplates a double stranded nucleic acid molecule comprising a nucleic acid molecule of the invention or an oligonucleotide fragment thereof hydrogen bonded to a complementary nucleotide base sequence.

The terms “isolated and purified nucleic acid” and “substantially pure nucleic acid”, e.g., substantially pure DNA, refer to a nucleic acid molecule which is one or both of the following: (1) not immediately contiguous with either one or both of the sequences, e.g., coding sequences, with which it is immediately contiguous (i.e., one at the 5′ end and one at the 3′end) in the naturally occurring genome of the organism from which the nucleic acid is derived; or (2) which is substantially free of a nucleic acid sequence with which it occurs in the organism from which the nucleic acid is derived. The term includes, for example, a recombinant DNA which is incorporated into a vector, e.g., into an autonomously replicating plasmid or virus, or into the genomic DNA of a prokaryote or eukaryote, or which exists as a separate molecule (e.g., a cDNA or a genomic DNA fragment produced by PCR or restriction endonuclease treatment) independent of other DNA sequences. Substantially pure or isolated and purified DNA also includes a recombinant DNA, which is part of a hybrid gene encoding additional HSLC7A11 sequence.

The invention provides in one embodiment: (a) an isolated and purified nucleic acid molecule comprising a sequence encoding all or a portion of a protein having the amino acid sequence as shown in SEQ ID NO: 4 or SEQ ID NO: 6; (b) nucleic acid sequences complementary to (a); (c) nucleic acid sequences which exhibit at least 80%, more preferably at least 90%, more preferably at least 95%, and most preferably at least 98% sequence identity to (a); or (d) a fragment of (a) or (b) that is at least 18 bases and which will hybridize to (a) or (b) under stringent conditions. In a particular embodiment, the fragment is a sequence encoding a hSLC7A11 having the amino acid sequence as shown in SEQ ID NO: 4 or SEQ ID NO: 6 and sequences having at least 80%, more preferably at least 85%, more preferably at least 90%, more preferably at least 95%, more preferably at least 96%, more preferably at least 97%, more preferably at least 98%, and most preferably at least 99% sequence identity thereto.

The degree of homology (percent identity) between a native and a mutant sequence may be determined, for example, by comparing the two sequences using computer programs commonly employed for this purpose. One suitable program is the GAP computer program described by Devereux et al., (1984) Nucl. Acids Res. 12:387. The GAP program utilizes the alignment method of Needleman and Wunsch (1970) J. Mol. Biol. 48:433, as revised by Smith and Waterman (1981) Adv. Appl. Math. 2:482. Briefly, the GAP program defines percent identity as the number of aligned symbols (i.e., nucleotides or amino acids) which are identical, divided by the total number of symbols in the shorter of the two sequences.

As used herein the term “stringent conditions” encompasses conditions known in the art under which a nucleotide sequence will hybridize to an isolated and purified nucleic acid molecule comprising a sequence encoding a protein having the amino acid sequence as shown herein, or to (b) a nucleic acid sequence complementary to (a). Screening polynucleotides under stringent conditions may be carried out according to the method described in Nature, 313:402-404 (1985). Polynucleotide sequences capable of hybridizing under stringent conditions with the polynucleotides of the invention may be, for example, allelic variants of the disclosed DNA sequences, or may be derived from other sources. General techniques of nucleic acid hybridization are disclosed by Sambrook et al., Molecular Cloning: A Laboratory Manual, 2nd Ed., Cold Spring Harbor Laboratory, Cold Spring Harbor, N.Y. (1984); and by Haymes et al., Nucleic Acid Hybridization: A Practical Approach, IRL Press, Washington, D.C. (1985), which references are incorporated herein by reference.

The invention also provides: (a) a purified and isolated nucleic acid molecule comprising a sequence as shown in SEQ ID NO: 1; (b) nucleic acid sequences complementary to (a); (c) nucleic acid sequences having at least 80%, more preferably at least 90%, more preferably at least 95%, and most preferably at least 98% sequence identity to (a); or (d) a fragment of (a) or (b) that is at least 18 bases and which will hybridize to (a) or (b) under stringent conditions.

The invention additionally includes nucleic acid molecules of the invention having one or more structural mutations including replacement, deletion, or insertion mutations. For example, a signal peptide may be deleted or conservative amino acid substitutions may be made to generate a protein that is still biologically competent or active.

The invention further includes a recombinant molecule comprising a nucleic acid molecule of the invention or an oligonucleotide fragment thereof and an expression control sequence operatively linked to the nucleic acid molecule or oligonucleotide fragment. A transformant host cell including a recombinant molecule of the invention is also provided.

In another aspect, the invention features a cell or purified preparation of cells which include a novel gene encoding a hSLC7A11 of the invention, or which otherwise misexpresses a gene encoding a hSLC7A11 of the invention. The cell preparation can consist of human or non-human cells, e.g., insect cells, rodent cells (e.g., mouse or rat cells), rabbit cells, or pig cells. In preferred embodiments, the cell or cells include a hSLC7A11 transgene, e.g., a heterologous form of a hSLC7A11 gene, e.g., a gene derived from humans (in the case of a non-human cell). The hSLC7A11 transgene can be misexpressed, e.g., overexpressed or underexpressed. In other preferred embodiments, the cell or cells include a gene that misexpresses an endogenous hSLC7A11 gene, e.g., a gene the expression of which is disrupted, e.g., a knockout. Such cells can serve as a model for studying disorders which are related to mutated or misexpressed hSLC7A11 alleles for use in drug screening.

Still further, the invention provides plasmids which comprise the nucleic acid molecules of the invention.

The invention also includes a novel hSLC7A11 of the invention, or an active part thereof. A biologically competent or active form of the protein or part thereof is also referred to herein as an “active hSLC7A11 or part thereof”.

The invention further contemplates antibodies having specificity against an epitope of the hSLC7A11 of the invention or part of the protein. These antibodies may be polyclonal or monoclonal. The antibodies may be labeled with a detectable substance and they may be used, for example, to detect the novel hSLC7A11 of the invention in tissue and cells. Additionally, the antibodies of the invention, or portions thereof, may be used to make targeted antibodies that destroy hSLC7A11 expressing cells (e.g., antibody-toxin fusion proteins, or radiolabelled antibodies).

The invention also permits the construction of nucleotide probes that encode part or all of the novel hSLC7A11 protein of the invention or a part of the protein. Thus, the invention also relates to a probe comprising a nucleotide sequence coding for a protein, which displays the properties of the novel hSLC7A11 of the invention or a peptide unique to the protein. The probe may be labeled, for example, with a detectable (e.g., radioactive) substance and it may be used to select from a mixture of nucleotide sequences a nucleotide sequence coding for a protein which displays the properties of the novel hSLC7A11 of the invention.

The invention also provides a transgenic insect or non-human animal (e.g., a rodent, e.g., a mouse or a rat, a rabbit, or a pig) or embryo all of whose germ cells and somatic cells contain a recombinant molecule of the invention, preferably a recombinant molecule comprising a nucleic acid molecule of the invention encoding the hSLC7A11 of the invention or part thereof. The recombinant molecule may comprise a nucleic acid sequence encoding the hSLC7A11 of the invention with a structural mutation, or may comprise a nucleic acid sequence encoding the hSLC7A11 of the invention or part thereof and one or more regulatory elements which differ from the regulatory elements that drive expression of the native protein. In another preferred embodiment, the insect or animal has a hSLC7A11 gene which is misexpressed or not expressed, e.g., a knockout. Such transgenic animals can serve as a model for studying disorders that are related to mutated or misexpressed hSLC7A11 of the invention.

The invention still further provides a method for identifying a substance which is capable of binding the novel hSLC7A11 of the invention, comprising reacting the novel hSLC7A11 of the invention or part of the protein under conditions which permit the formation of a complex between the substance and the novel hSLC7A11 protein or part of the protein, and assaying for substance-hSLC7A11 complexes, for free substance, for non-complexed hSLC7A11, or for activation of the substance (e.g., receptor) that binds to the hSLC7A11 of the invention.

Another aspect of the invention is a method for identifying receptors which are capable of binding the hSLC7A11 proteins of the invention, including isoforms and fragments, said method comprising reacting a hSLC7A11 protein of the invention, or an isoform or fragment thereof, with at least one receptor which potentially is capable of binding to the protein, isoform, or part of the protein, under conditions which permit the formation of receptor-ligand protein complexes, and assaying for receptor-ligand protein complexes, for free hSLC7A11 for non-complexed receptor protein, or for activation of the receptor that binds to the hSLC7A11 of the invention. In a preferred embodiment of the method, receptors are identified which are capable of binding the novel hSLC7A11 protein of the invention, isoforms thereof, or part of the protein.

The invention also relates to a method for assaying a medium for the presence of an agonist or antagonist of the interaction of the novel hSLC7A11 protein and a substance which is capable of binding the hSLC7A11 said method comprising providing a known concentration of the hSLC7A11, reacting the hSLC7A11 with a substance (e.g., receptor) which is capable of binding the hSLC7A11 and a suspected agonist or antagonist under conditions which permit the formation of substance-hSLC7A11 complexes, and assaying for substance-hSLC7A11 complexes, for free substance, for non-complexed hSLC7A11, or for activation of the substance (e.g., receptor).

Also included within the scope of the invention is a composition which includes the hSLC7A11 of the invention, a fragment thereof (or a nucleic acid encoding said hSLC7A11 or fragment thereof) and one or more additional components, e.g., a carrier, diluent or solvent. The additional component can be one which renders the composition useful for in vitro, in vivo, pharmaceutical, or veterinary use.

In another aspect, the invention relates to a method of treating a mammal, e.g., a human, at risk for a disorder, e.g., a disorder characterized by aberrant or unwanted level or biological activity of the hSLC7A11 of the invention, or characterized by an aberrant or unwanted level of a ligand that specifically binds the hSLC7A11 of the invention. For example, the hSLC7A11 of the invention may be useful to leach out or block a ligand that is found to bind to the hSLC7A11 of the invention.

The full-length and splice variant cDNA sequences for the coding region of human SLC7A11 were cloned using Ref Seq NM_{—}014331 as a reference to design the following oligonucleotides:

| | |
|---|---|
| SLC7A11-PCR1: | |
| CACCGAATTCTGTGTCCCTACTATGTCAGAAAGCCTG | (SEQ ID NO:1) |
| TTGTG | |
| SLC7A11-PCR2: | |
| TAACTTATCTTCTTCTGGTACAACTTCCAGTATTATT | (SEQ ID NO:2) |
| TGTAATGTTCTGG | |

PCR conditions were: 95° C. denaturing temperature for 30 minutes annealing using a temperature gradient thermocycler (Eppendorf Mastercycler) with a range of 50° C. to 70° C. for one hour and 30 minutes, followed by synthesis at 72° C. for two hours and 30 minutes. A mixture of cDNAs from different sources (cancer cell lines, human spleen, brain, placenta, liver) was used as a template and Pfu polymerase (Stratagene) as enzyme in the presence of 10% DMSO, 250 μM dNTPs, 1×Pfu reaction buffer. The resulting PCR product was gel purified and cloned using the “pENTR Directional TOPO Cloning Kit” from Invitrogen, and several independent clones were sequenced. Two cDNA products were identified, one representing a splice product which encodes a shorter version of the hSLC7A11 peptide having a different C-terminus, i.e. missing the last five transmembrane domains.

The sequences for the two identified hSLC7A11 clones are as follows:

SLC7A11 Full Length DNA Sequence

(SEQ ID NO:3)

CACCGAATTCTGTGTCCCTACTATGGTCAGAAAGCCTGTTGTGTCCACCA
TCTCCAAAGGAGGTTACCTGCAGGGAAATGTTAACGGGAGGCTGCCTTCC
CTGGGCAACAAGGAGCCACCTGGGCAGGAGAAAGTGCAGCTGAAGAGGAA
AGTCACTTTACTGAGGGGAGTCTCCATTATCATTGGCACCATCATTGGAG
CAGGAATCTTCATCTCTCCTAAGGGCGTGCTCCAGAACACGGGCAGCGTG
GGCATGTCTCTGACCATCTGGACGGTGTGTGGGGTCCTGTCACTATTTGG
AGCTTTGTCTTATGCTGAATTGGGAACAACTATAAAGAAATCTGGAGGTC
ATTACACATATATTTTGGAAGTCTTTGGTCCATTACCAGCTTTTGTACGA
GTCTGGGTGGAACTCCTCATAATACGCCCTGCAGCTACTGCTGTGATATC
CCTGGCATTTGGACGCTACATTCTCGAACCATTTTTTATTCAATGTGAAA
TCCCTGAACTTGCGATCAAGCTCATTACAGCTGTGGGCATAACTGTAGTG
ATGGTCCTAAATAGCATGAGTGTCAGCTGGAGCGCCCGGATCCAGATTTT
CTTAACCTTTTGCAAGCTCACAGCAATTCTGATAATTATAGTCCCTGGAG
TTATGCAGCTAATTAAAGGTCAAACGCAGAACTTTAAAGACGCCTTTTCA
GGAAGAGATTCAAGTATTACGCGGTTGCCACTGGCTTTTTATTATGGAAT
GTATGCATATGCTGGCTGGTTTTACCTCAACTTTGTTACTGAAGAAGTAG
AAAACCCTGAAAAAACCATTCCCCTTGCAATATGTATATCCATGGCCATT
GTCACCATTGGCTATGTGCTGACAAATGTGGCCTACTTTACGACCATTAA
TGCTGAGGAGCTGCTGCTTTCAAATGCAGTGGCAGTGACCTTTTCTGAGC
GGCTACTGGGAAATTTCTCATTAGCAGTTCCGATCTTTGTTGCCCTCTCC
TGCTTTGGCTCCATGAACGGTGGTGTGTTTGCTGTCTCCAGGTTATTCTA
TGTTGCGTCTCGAGAGGGTCACCTTCCAGAAATCCTCTCCATGATTCATG
TCCGCAAGCACACTCCTCTACCAGCTGTTATTGTTTTGCACCCTTTGACA
ATGATAATGCTCTTCTCTGGAGACCTCGACAGTCTTTTGAATTTCCTCAG
TTTTGCCAGGTGGCTTTTTATTGGGCTGGCAGTTGCTGGGCTGATTTATC
TTCGATACAAATGCCCAGATATGCATCGTCCTTTCAAGGTGCCACTGTTC
ATCCCAGCTTTGTTTTCCTTCACATGCCTCTTCATGGTTGCCCTTTCCCT
CTATTCGGACCCATTTAGTACAGGGATTGGCTTCGTCATCACTCTGACTG
GAGTCCCTGCGTATTATCTCTTTATTATATGGGACAAGAAACCCAGGTGG
TTTAGAATAATGTCGGAGAAAATAACCAGAACATTACAAATAATACTGGA
AGTTGTACCAGAAGAAGATAAGTTATGA
SLC7A11 Full Length Peptide Sequence

(SEQ ID NO:4)

MVRKPVVSTISKGGYLQGNVNGRLPSLGNKEPPGQEKVQLKRKVTLLRGV
SILIGTIIGAGIFISPKGVLQNTGSVGMSLTIWTVCGVLSLFGALSYAEL
GTTIKKSGGHYTYILEVFGPLPAFVRVWVELLIIRPAATAVISLAFGRYI
LEPFFIQCEIPELAIKLITAVGITVVMVLNSMSVSWSARIQIFLTFCKLT
AILIIIVPGVMQLIKGQTQNFKDAFSGRDSSITRLPLAFYYGMYAYAGWF
YLNFVTEEVENPEKTIPLAICLSMAIVTIGYVLTNVAYFTTTNAEELLLS
NAVAVTFSERLLGNFSLAVPIFVALSCFGSMNGGVFAVSRLFYVASREGH
LPELLSMIHVRKFITPLPAVIVLHPLTMIMLFSGDLDSLLNFLSFARWLF
IGLAVAGLIYLRYKCPDMHRPFKVPLFIPALFSFTCLFMVALSLYSDPFS
TGIGFVITLTGVPAYYLFIIWDKKPRWFRIMSEKITRTLQIILEVVPEED
KL
SLC7A11_Splice Variant DNA Sequence

(SEQ ID NO:5)

CACCGAATTCTGTGTCCCTACTATGGTCAGAAAGCCTGTTGTGTCCACCA
TCTCCAAAGGAGGTTACCTGCAGGGAAATGTTAACGGGAGGCTGCCTTCC
CTGGGCAACAAGGAGCCACCTGGGCAGGAGAAAGTGCAGCTGAAGAGGAA
AGTCACTTTACTGAGGGGAGTCTCCATTATCATTGGCACCATCATTGGAG
CAGGAATCTTCATCTCTCCTAAGGGCGTGCTCCAGAACACGGGCAGCGTG
GGCATGTCTCTGACCATCTGGACGGTGTGTGGGGTCCTGTCACTATTTGG
AGCTTTGTCTTATGCTGAATTGGGAACAACTATAAAGAAATCTGGAGGTC
ATTACACATATATTTTGGAAGTCTTTGGTCCATTACCAGCTTTTGTACGA
GTCTGGGTGGAACTCCTCATAATACGCCCTGCAGCTACTGCTGTGATATC
CCTGGCATTTGGACGCTACATTCTGGAACCATTTTTTATTCAATGTGAAA
TCCCTGAACTTGCGATCAAGCTCATTACAGCTGTGGGCATAACTGTAGTG
ATGGTCCTAAATAGCATGAGTGTCAGCTGGAGCGCCCGGATCCAGATTTT
CTTAACCTTTTGCAAGCTCACAGCAATTCTGATAATTATAGTCCCTGGAG
TTATGCAGCTAATTAAAGGTCAAACGCAGAACTTTAAAGACGCCTTTTCA
GGAAGAGATTCAAGTATTACGCGGTTGCCACTGGCTTTTTATTATGGAAT
GTATGCATATGCTGGCTGGTTTTACCTCAACTTTGTTACTGAAGAAGTAG
AAAACCCTGAAAAAACCATTCCCCTTGCAATATGTATATCCATGGCCATT
GTCACCATTGGCTATGTGCTGACAAATGTGGCCTACTTTACGACCATTAA
TGCTGAGGAGCTGCTGCTTTCAAATGCAGTGGCAGTGACCTTTTCTGAGC
GGCTACTGGGAAATTTCTCATTAGCAGTTCCGATCTTTGTTGCCCCCTCC
TCTACCAGCTGTTATTGTTTTGCACCCTTTGACAATGATAATGCTCTTCT
CTGGAGACCTCGACAGTCTTTTGAATTTCCTCAGTTTTGCCAGGTGGCTT
TTTATTGGGCTGGCAGTTGCTCGGCTGATTTATCTTCGATACAAATGCCC
AGATATGCATCGTCCTTTCAAGGTGCCACTGTTCATCCCAGCTTTGTTTT
CCTTCACATGCCTCTTCATGGTTGCCCTTTCCCTCTATTCGGACCCATTT
AGTACAGGGATTGGCTTCGTCATCACTCTGACTGGAGTCCCTGCGTATTA
TCTCTTTATTATATGGGACAAGAAACCCAGGTGGTTTAGAATAATGTCGG
AGAAAATAACCAGAACATTACAAATAATACTGGAAGTTGTACCAGAAGAA
GATAAGTTA
SLC7A11_Splice Variant Peptide Sequence

(SEQ ID NO:6)

MVRKPVVSTISKGGYLQGNVNGRLPSLGNKEPPGQEKVQLKRKVTLLRGV
SIIIGTIIGAGIFISPKGVLQNTGSVGMSLTIWTVCGVLSLFGALSYAEL
GTTIKKSGGHYTYILEVFGPLPAFVRVWVELLTTRPAATAVISLAFGRYI
LEPFFIQCEIPELAIKLITAVGITVVMVLNSMSVSWSARIQIFLTFCKLT
AILIIIVPGVMQLIKGQTQNFKDAFSGRDSSITRLPLAFYYGMYAYAGWF
YLNFVTEEVENPEKTIPLAICISMAIVTIGYVLTNVAYFTTTNAEELLLS
NAVAVTFSERLLGNFSLAVPIFVAPSSTSCYCFAPFDNDNALLWRPRQSF
EFPQFCQVAFYWAGSCWADLSSIQMPRYASSFQGATVHPSFVFLHMPLHG
CPFPLFGPI

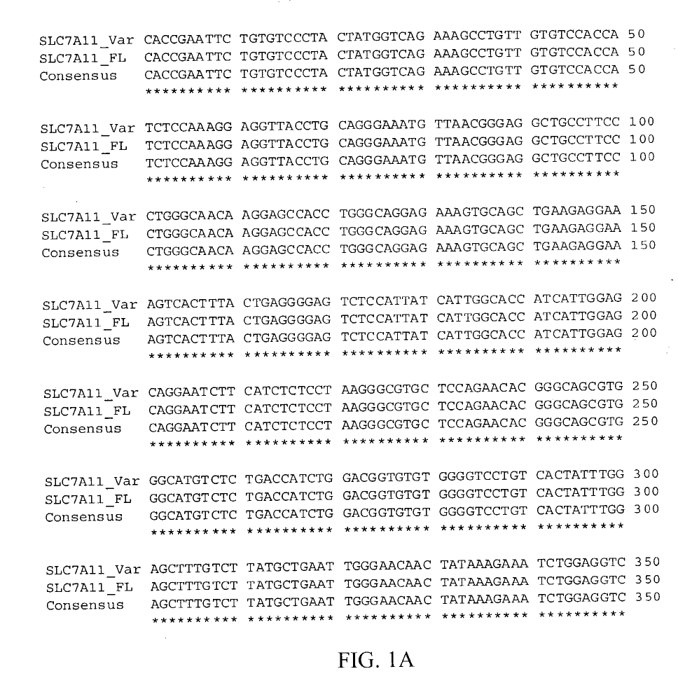

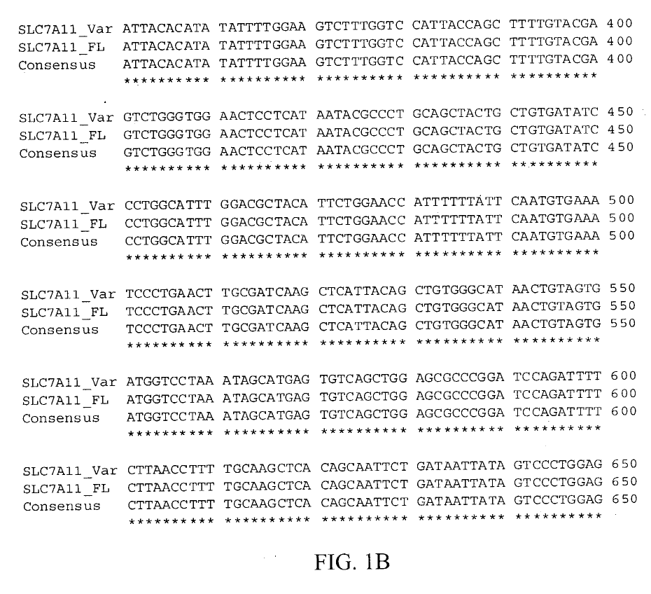

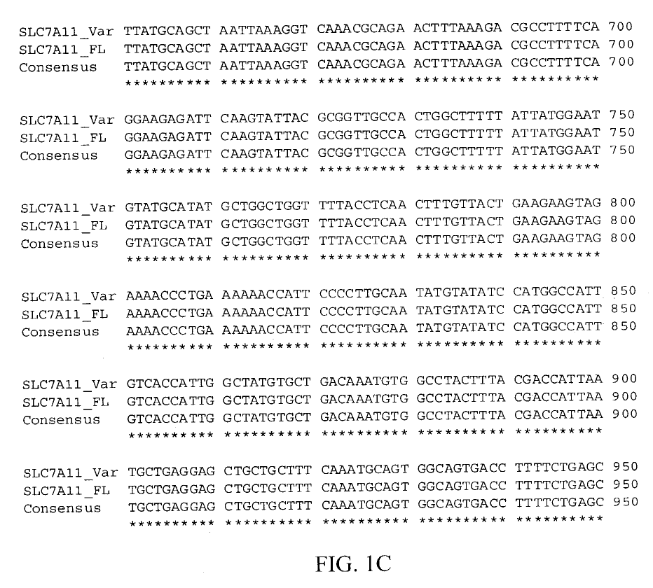

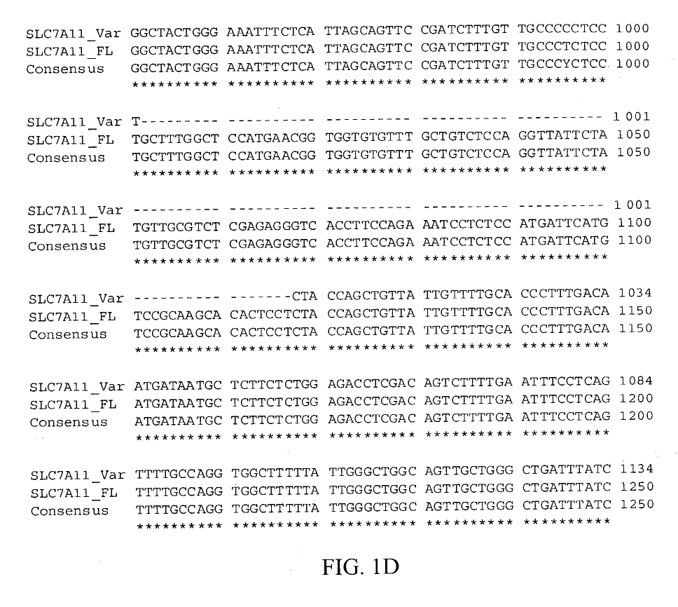

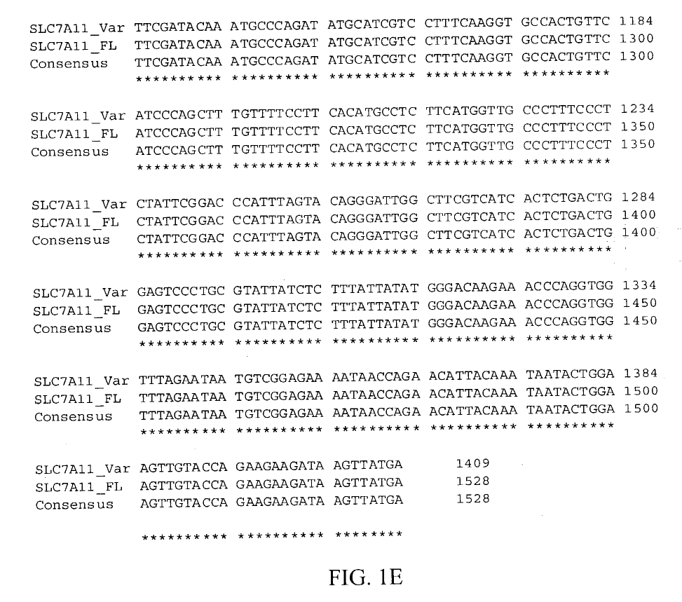

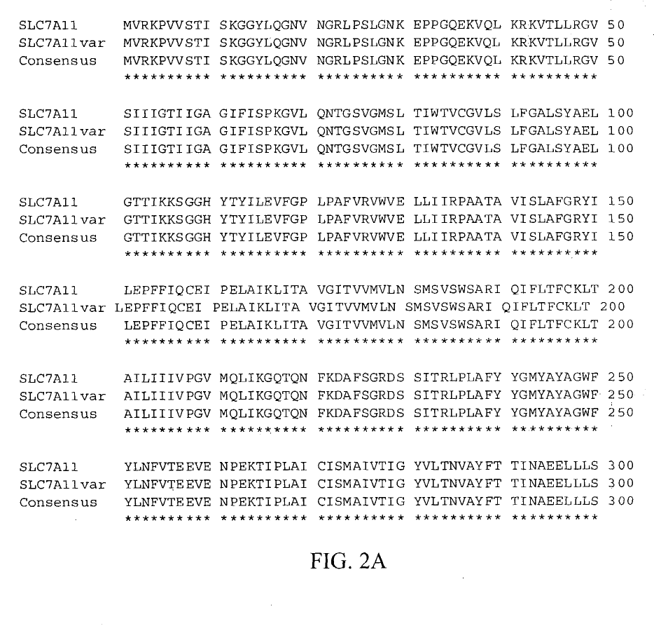

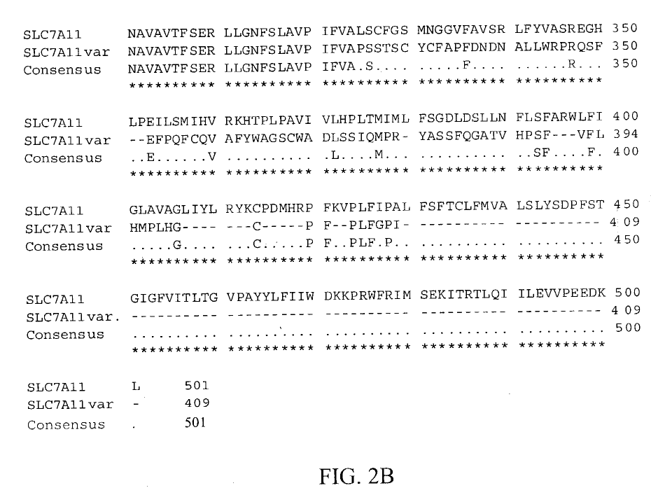

Alignment of the full length hSLC7A11 cDNA sequence (SEQ ID NO: 3) with that for the splice variant (SEQ ID NO: 5) is shown in FIGS. 1A-1E. FIGS. 2A-B show the corresponding alignment of the amino acid sequences. As shown therein, the splice variant is truncated in that it is missing five transmember domains in C terminus region.

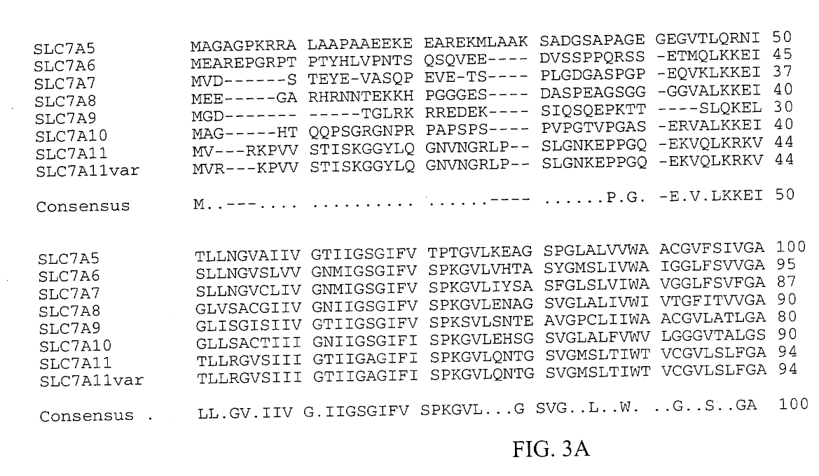

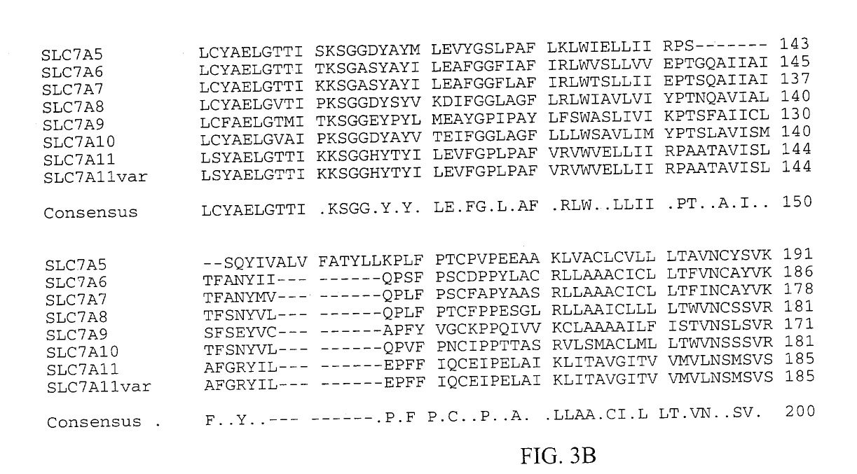

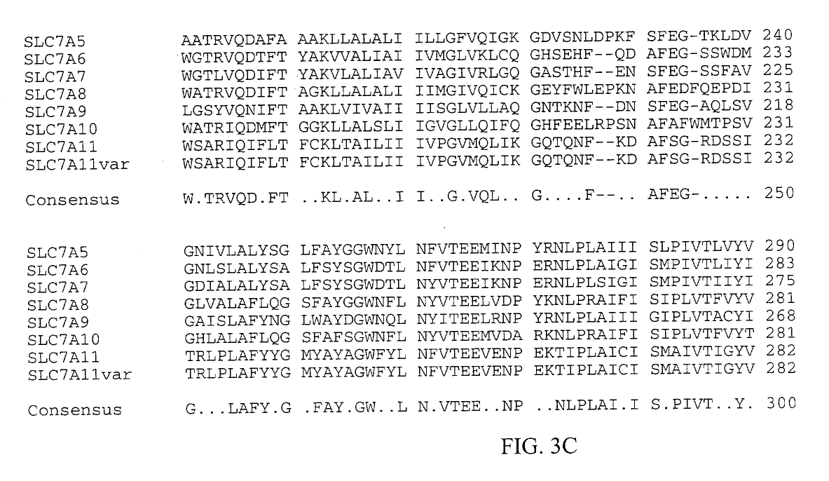

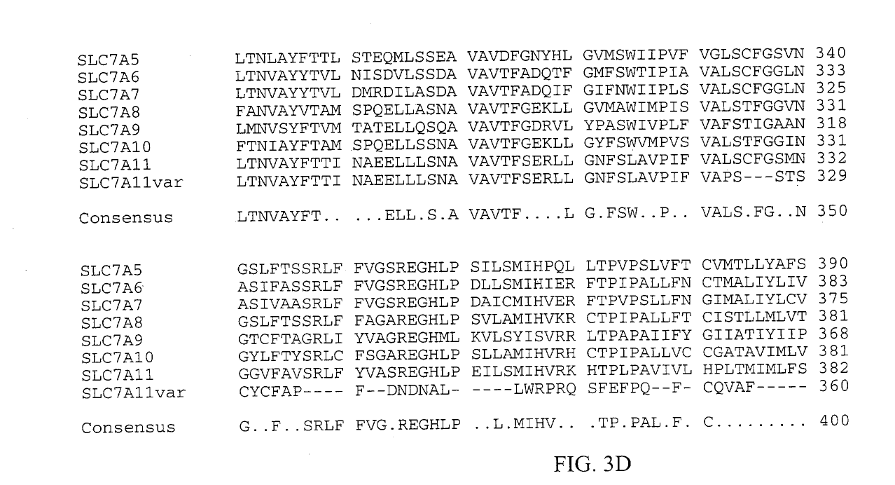

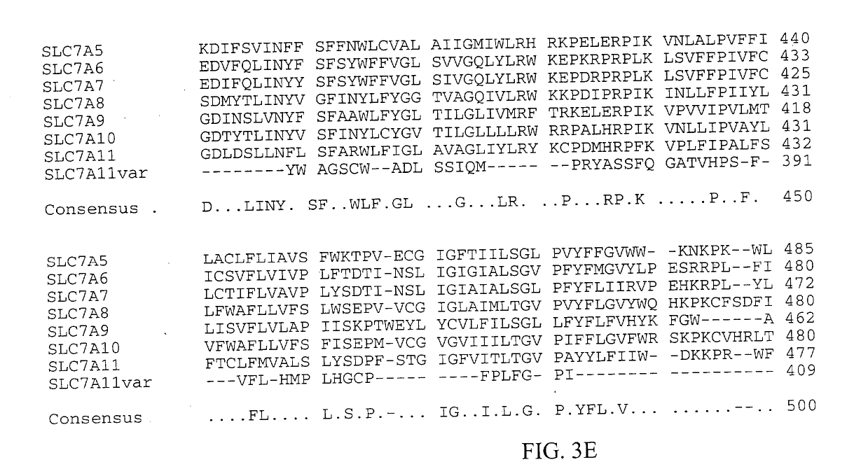

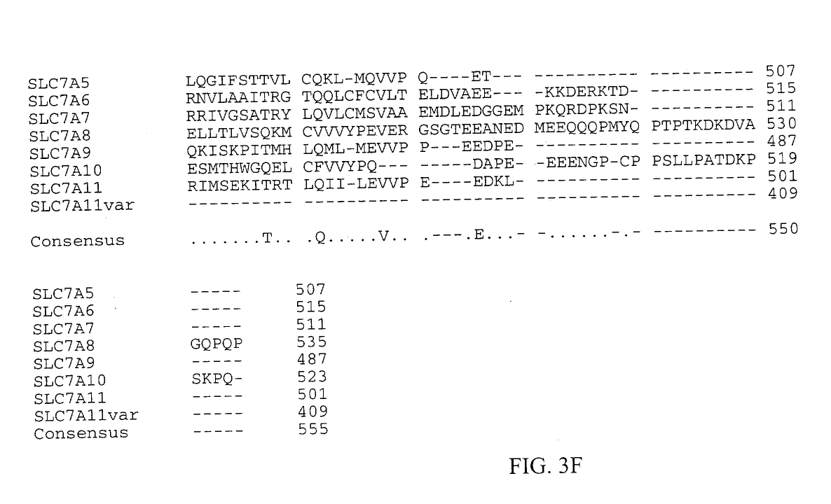

FIGS. 3A-F show the alignment of the amino acid sequence for full length hSLC7A11 (SEQ ID NO: 4) and hSLC7A11 splice variant (SEQ ID NO: 6) with other members of the SLC7 family. This alignment illustrates the similarities and characteristics denoting members of this family of genes.

The invention relates to nucleic acid sequences or a fragment thereof (referred to herein as a “polynucleotide”) of the novel hSLC7A11 as shown above (SEQ ID NO: 3 and SEQ ID NO: 5)), as well as to the amino acid sequences of hSLC7A11 (SEQ ID NO: 4 and SEQ ID NO; 6), and biologically active portions thereof.

The invention further relates to variants of the hereinabove described nucleic acid sequences which encode for fragments, analogs and derivatives of the polypeptides having the deduced amino acid sequences of SEQ ID NO: 4 and SEQ ID NO: 6. The variants of the nucleic acid sequence may be naturally occurring variants of the nucleic acid sequence or non-naturally occurring variants of the nucleic acid sequence.

Thus, the invention includes polynucleotides encoding the same mature polypeptides as shown in SEQ ID NO: 4 and SEQ ID NO: 6, as well as variants of such polynucleotides which variants encode for a fragment, derivative, or analog of the polypeptides of SEQ ID NO: 4 and SEQ ID NO: 6. Such nucleotide variants include deletion variants, substitution variants, and addition or insertion (splice) variants.

The term “gene” means the segment of DNA involved in producing a polypeptide chain; it includes regions preceding and following the coding region (leader and trailer) as well as intervening sequences (introns) between individual coding segments (exons).

Fragments of the full-length gene of the invention may be used as hybridization probes for a cDNA library to isolate the full-length gene and to isolate other genes which have a high sequence similarity to a gene of the invention or similar biological activity. Probes of this type preferably have at least between 20 and 30 bases, and may contain, for example, 50 or more bases. The probes may also be used to identify a cDNA clone corresponding to a full length transcript and a genomic clone or clones that contain the complete gene of the invention including regulatory and promoter regions, exons, and introns.

The invention further relates to polynucleotides that hybridize to the polynucleotide sequences disclosed herein, if there is at least 80%, preferably at least 90%, and more preferably at least 95% identity between the sequences. The invention particularly relates to polynucleotides which hybridize under stringent conditions to the polynucleotides described herein.

Alternatively the polynucleotide may have at least 20 bases, preferably at least 30 bases, and more preferably at least 50 bases which hybridize to a polynucleotide of the invention and which has an identity thereto, as hereinabove described, and which may or may not retain activity. For example, such polynucleotides may be employed as probes for the polynucleotide of SEQ ID NO: 1, for example for recovery of the polynucleotide or as a diagnostic probe or as a PCR primer.

Thus the invention is directed to polynucleotides having at least 80% identity, preferably at least 90% and more preferably at least 95% identity to a polynucleotide of the invention, including polynucleotides encoding the polypeptides of SEQ ID NO: 4 and SEQ ID NO: 6, as well as fragments thereof, which fragments have at least 20 or 30 bases, and preferably at least 50 bases, and to polypeptides encoded by such polynucleotides.

The invention further relates to a solute carrier family 7, member 11 molecule polypeptide, hSLC7A11 which has the deduced amino acid sequences as shown in SEQ ID NO: 4 and SEQ ID NO: 6, as well as fragments, analogs and derivatives of such polypeptide.

Analogs of the novel hSLC7A11 of the invention are also within the scope of the invention. Analogs can differ from the naturally occurring hSLC7A11 of the invention in amino acid sequence or in ways that do not involve sequence, or both. Non-sequence modifications include in vivo or in vitro chemical derivitization of the hSLC7A11 of the invention. Non-sequence modifications include changes in acetylation, methylation, phosphorylation, carboxylation, or glycosylation.

Preferred analogs include the novel hSLC7A11 of the invention (or biologically active fragments thereof) whose sequences differ from the wild-type sequences by one or more conservative amino acid substitutions or by one or more non-conservative amino acid substitutions, deletions, or insertions which do not abolish the biological activity of the hSLC7A11 of the invention. Conservative substitutions typically include the substitution of one amino acid for another with similar characteristics, e.g., substitutions within the following groups: valine, glycine; glycine, alanine; valine, isoleucine, leucine; aspartic acid, glutamic acid; asparagine, glutamine; serine, threonine; lysine, arginine; and phenylalanine, tyrosine. Other conservative amino acid substitutions can be taken from the table below.

TABLE 1

| For Amino Acid | Code | Replace with any of: |
|---|---|---|
| Alanine | A | D-Ala, Gly, beta-Ala, L-Cys, D-Cys |
| Arginine | R | D-Arg, Lys, D-Lys, homo-Arg, D-homo-Arg, |
| | | Met,Ile, D-Met, D-Ile, Orn, D-Orn |
| Asparagine | N | D-Asn, Asp, D-Asp, Glu, D-Glu, Gln, D-Gln |
| Aspartic Acid | D | D-Asp, D-Asn, Asn, Glu, D-Glu, Gln, D-Gln |
| Cysteine | C | D-Cys, S-Me-Cys, Met, D-Met, Thr, D-Thr |
| Glutamine | Q | D-Gln, Asn, D-Asn, Glu, D-Glu, Asp, D-Asp |
| Glutamic Acid | E | D-Glu, D-Asp, Asp, Asn, D-Asn, Gln, D-Gln |
| Glycine | G | Ala, D-Ala, Pro, D-Pro, β-Ala, Acp |
| Isoleucine | I | D-Ile, Val, D-Val, Leu, D-Leu, Met, D-Met |
| Leucine | L | D-Leu, Val, D-Val, Met, D-Met |
| Lysine | K | D-Lys, Arg, D-Arg, homo-Arg, D-homo-Arg, |
| | | Met, D-Met, Ile, D-Ile, Orn, D-Orn |
| Methionine | M | D-Met, S-Me-Cys, Ile, D-Ile, Leu, D-Leu, |
| | | Val, D-Val |
| Phenylalanine | F | D-Phe, Tyr, D-Thr, L-Dopa, His, D-His, |
| | | Trp, D-Trp, Trans-3, 4, or 5-phenylproline, cis- |
| | | 3, 4, or 5-phenylproline |
| Proline | P | D-Pro, L-1-thioazolidine-4-carboxylic acid, |
| | | D- or L-1-oxazolidine-4-carboxylic acid |
| Serine | S | D-Ser, Thr, D-Thr, allo-Thr, Met, D-Met, |
| | | Met(o), D-Met(O), L-Cys, D-Cys |
| Threonine | T | D-Thr, Ser, D-Ser, allo-Thr, Met, D-Met, |
| | | Met(O), D-Met(O), Val, D-Val |
| Tyrosine | Y | D-Tyr, Phe, D-Phe, L-Dopa, His, D-His |
| Valine | V | D-Val, Leu, D-Leu, Ile, D-Ile, Met, D-Met |

Other analogs within the invention are those with modifications which increase protein or peptide stability; such analogs may contain, for example, one or more non-peptide bonds (which replace the peptide bonds) in the protein or peptide sequence. Also included are analogs that include residues other than naturally occurring L-amino acids, e.g., D-amino acids or non-naturally occurring or synthetic amino acids, e.g., β or γ amino acids.

In terms of general utility of the hSLC7A11 of the invention, gene expression of hSLC7A11 suggests it is important in human cancers. Such a cancer may include, but is not limited to, adenocarcinoma, leukemia, lymphoma, melanoma, myeloma, sarcoma, teratocarcinoma and, in particular, cancers of the adrenal gland, bladder, bone, bone marrow, brain, breast, cervix, colon, gall bladder, ganglia, gastrointestinal tract, heart, kidney, liver, lung, muscle, ovary, pancreas, parathyroid, penis, prostrate, salivary glands, skin, spleen, testis, thymus, throid and uterus. As such, any of the proteins, antagonists, antibodies, agonists, complementary sequences, or vectors of the invention may be administered to a subject to treat or prevent a cancer.

Gene constructs of the invention can also be used as part of a gene therapy protocol to deliver nucleic acids encoding the hSLC7A11 of the invention, or an agonist or antagonist form of a hSLC7A11 protein or peptide. The invention features expression vectors for in vivo transfection and expression of a hSLC7A11. Expression constructs of the hSLC7A11 of the invention, may be administered in any biologically effective carrier, e.g., any formulation or composition capable of effectively delivering the hSLC7A11 gene to cells in vivo. Approaches include insertion of the subject gene in viral vectors including recombinant retroviruses, adenoviruses, adeno-associated viruses, and herpes simplex virus-1, or recombinant bacterial or eukaryotic plasmids. Viral vectors transfect cells directly; an advantage of infection of cells with a viral vector is that a large proportion of the targeted cells can receive the nucleic acid. Several viral delivery systems are known in the art and can be utilized by one practicing the invention.

In addition to viral transfer methods, non-viral methods may also be employed to cause expression of the hSLC7A11 in the tissue of an insect or animal. Most non-viral methods of gene transfer rely on normal mechanisms used by mammalian cells for the uptake and intracellular transport of macromolecules. Exemplary gene delivery systems of this type include liposomal derived systems, poly-lysine conjugates, and artificial viral envelopes. DNA of the invention may also be introduced to cell(s) by direct injection of the gene construct or electroporation.

In clinical settings, the gene delivery systems for the therapeutic hSLC7A11 gene (or homologue thereof identified using all or a portion of the gene disclosed herein) can be introduced into a patient by any of a number of methods, each of which is known in the art. For instance, a pharmaceutical preparation of the gene delivery system can be introduced systemically, e.g., by intravenous injection, and specific transduction of the protein in the target cells occurs predominantly from specificity of transfection provided by the gene delivery vehicle, cell-type or tissue-type expression due to the transcriptional regulatory sequences controlling expression of the receptor gene, or a combination thereof.

The pharmaceutical preparation of the gene therapy construct can consist essentially of the gene delivery system in an acceptable diluent, or can comprise a slow release matrix in which the gene delivery vehicle is embedded. Alternatively, where the complete gene delivery system can be produced intact from recombinant cells, e.g., retroviral vectors, the pharmaceutical preparation can comprise one or more cells which produce the gene delivery system.

In other embodiments, any of the proteins, antagonists, antibodies, agonists, complementary sequences, or vectors of the invention may be administered in combination with other appropriate therapeutic agents. Selection of the appropriate agents for use in combination therapy may be made by one of ordinary skill in the art, according to conventional pharmaceutical principles. The combination of therapeutic agents may act synergistically to effect the treatment or prevention

Another aspect of the invention relates to the use of an isolated nucleic acid in antisense therapy. As used herein, antisense therapy refers to administration or in situ generation of oligonucleotides or their derivatives which specifically hybridize under cellular conditions, with the cellular mRNA and/or genomic DNA encoding the HSLC7A11 of the invention so as to inhibit expression of the encoded protein, e.g., by inhibiting transcription and/or translation. In general, antisense therapy refers to the range of techniques generally employed in the art, and includes any therapy which relies on specific binding to oligonucleotide sequences.

Fragments of the hSLC7A11 of the invention are also within the scope of the invention. Fragments of the protein can be produced in several ways, e.g., recombinantly, by proteolytic digestion, or by chemical synthesis. Internal or terminal fragments of a polypeptide can be generated by removing one or more nucleotides from one end (for a terminal fragment) or both ends (for an internal fragment) of a nucleic acid which encodes the polypeptide. Digestion with “end-nibbling” endonucleases can thus generate DNAs which encode an array of fragments. DNAs which encode fragments of the hSLC7A11 protein can also be generated by random shearing, restriction digestion, or a combination of the above-discussed methods.

Fragments can also be chemically synthesized using techniques known in the art such as conventional Merrifield solid phase f-Moc or t-Boc chemistry.

Amino acid sequence variants of the hSLC7A11 protein of the invention can be prepared by random mutagenesis of DNA which encodes a protein or a particular domain or region of the protein. Useful methods are known in the art, e.g., PCR mutagenesis and saturation mutagenesis. A library of random amino acid sequence variants can also be generated by the synthesis of a set of degenerate oligonucleotides sequences, a process known and practiced by those skilled in the art.

Non-random or directed mutagenesis techniques can be used to provide specific sequences or mutations in specific regions. These techniques can be used to create variants, which include, e.g., deletions, insertions, or substitutions of residues of the known amino acid sequence of the hSLC7A11 protein of the invention. The sites for mutation can be modified individually or in series, e.g., by (1) substituting first with conserved amino acids then with more radical choices depending upon results achieved; (2) deleting the target residue; or (3) inserting residues of the same or a different class (e.g., hydrophobic or hydrophilic) adjacent to the located site, or a combination of options (1)-(3). Alanine scanning mutagenesis is a useful method for identification of certain functional residues or regions of a desired protein that are preferred locations or domains for mutagenesis. Oligonucleotide-mediated mutagenesis, cassette mutagenesis, and combinatorial mutagenesis are useful methods known to those skilled in the art for preparing substitution, deletion, and insertion variants of DNA.

The invention also relates to methods of screening. Various techniques are known in the art for screening generated mutant gene products. Techniques for screening large gene libraries often include cloning the gene library into replicable expression vectors, transforming appropriate cells with the resulting library of vectors, and expressing the genes under conditions in which detection of a desired activity, e.g., in this case binding of the hSLC7A11 of the invention to its receptor. Techniques known in the art are amenable to high through-put analysis for screening large numbers of sequences created, e.g., by random mutagenesis techniques.

Two hybrid assays can be used to identify modulators of the interaction between a receptor and the hSLC7A11 of the invention. These modulators may include agonists or antagonists. In one approach to screening assays, the candidate protein or peptides are displayed on the surface of a cell or viral particle, and the ability of particular cells or viral particles to bind an appropriate receptor protein via the displayed product is detected in a “panning assay”. In a similar fashion, a detectably labeled ligand can be used to score for potentially functional peptide homologues. Fluorescently labeled ligands, e.g., receptors, can be used to detect homologue which retain ligand-binding activity. The use of fluorescently labeled ligand allows cells to be visually inspected and separated under fluorescence microscope or to be separated by a fluorescence-activated cell sorter.

High through-put assays can be followed by secondary screens in order to identify further biological activities which will, for example, allow one skilled in the art to differentiate agonists from antagonists. The type of a secondary screen used will depend on the desired activity that needs to be tested. For example, an assay can be developed in which the ability to inhibit an interaction between a receptor and the hSLC7A11 of the invention can be used to identify antagonists from a group of peptide fragments isolated through one of the primary screens. Therefore, methods for generating fragments and analogs and testing them for activity are known in the art. Once a sequence of interest is identified, it is routine for one skilled in the art to obtain agonistic or antagonistic analogs, fragments, and/or ligands.

Drug screening assays are also provided in the invention. By producing purified and recombinant hSLC7A11 of the invention, or fragments thereof, one skilled in the art can use these to screen for drugs which are either agonists or antagonists of the normal cellular function or their role in cellular signaling. In one embodiment, the assay evaluates the ability of a compound to modulate binding between a receptor and the hSLC7A11 of the invention. The term “modulating” encompasses enhancement, diminishment, activation, or inactivation of the receptor for hSLC7A11. Assays useful to identify a receptor to the hSLC7A11 of the invention are encompassed herein. A variety of assay formats will suffice and are known by those skilled in the art.

In many drug screening programs which test libraries of compounds and natural extracts, high throughput assays are desirable in order to maximize the number of compounds surveyed in a given period of time. Assays which are performed in cell-free systems, such as may be derived with purified or semi-purified proteins, are often preferred as primary screens in that they can be generated to permit rapid development and relatively easy detection of an alteration in a molecular target which is mediated by a test compound.

Also within the scope of the invention is a process for modulating the activity of the hSLC7A11 of the invention, directly or through the receptor for the hSLC7A11 disclosed herein. The term “modulating” encompasses enhancement, diminishment, activation, or inactivation of the activity of the hSLC7A11 disclosed herein. Ligands to the receptor of the hSLC7A11 of the invention, including peptides, proteins, small molecules, and antibodies, that are capable of binding to the receptor and modulating its activity are encompasses herein. Also encompassed herein are molecules that bind to the hSLC7A11 disclosed herein (e.g., antibodies specific for the hSLC7A11 of the invention). These compounds are useful in modulating the activity of the hSLC7A11 and/or the receptor for hSLC7A11, and in treating hSLC7A11-associated disorders. “hSLC7A11-associated disorders” refers to any disorder or disease state in which the hSLC7A11 protein plays a regulatory role in the metabolic pathway of that disorder or disease. Such disorders or diseases may include the cancer, as described above. As used herein the term “treating” refers to the alleviation of symptoms of a particular disorder in a patient, the improvement of an ascertainable measurement associated with a particular disorder, or the prevention of a particular immune, inflammatory, or cellular response (such as transplant rejection).

The invention also includes antibodies specifically reactive with the hSLC7A11 of the invention, or a portion thereof. Anti-protein/anti-peptide antisera or monoclonal antibodies can be made by standard known procedures. A mammal such as a mouse, hamster, or rabbit can be immunized with an immunogenic form of the peptide. Techniques for conferring immunogenicity on a protein or peptide include conjugation to carriers or other techniques known in the art. An immunogenic portion of the hSLC7A11 of the invention can be administered in the presence of adjuvant. The progress of immunization can be monitored by detection of antibody titers in plasma or serum.

The term “antibody” as used herein is intended to include fragments thereof which are also specifically reactive with the hSLC7A11 of the invention. Antibodies can be fragmented using conventional techniques and the fragments screened for utility in the same manner as whole antibodies. For example, F(ab′)2 fragments can be generated by treating antibody with pepsin. The resulting F(ab′)2 fragment can be treated to reduce disulfide bridges to produce Fab′ fragments. The antibody of the invention is further intended to include chimeric and humanized molecules that recognize and bind to the hSLC7A11 of the invention.

Both monoclonal and polyclonal antibodies directed against the hSLC7A11 of the invention, and antibody fragments such as Fab′, sFv and F(ab′)2, can be used to block the action of the hSLC7A11 of the invention and allow study of the role of a particular hSLC7A11 of the invention. Alternatively, such antibodies can be used therapeutically to block the hSLC7A11 of the invention in a subject mammal, e.g., a human. In a preferred embodiment a therapeutic composition comprising an antibody of the invention can also comprise a pharmaceutically acceptable carrier, solvent or diluent, and be administered by systems known in the art.

Antibodies that specifically bind to the hSLC7A11 of the invention, or fragments thereof, can also be used in immunohistochemical staining of tissue samples in order to evaluate the abundance and pattern expression of the hSLC7A11 of the invention. Antibodies can be used diagnostically in immunoprecipitation, immunoblotting, and enzyme linked immunosorbent assay (ELISA) to detect and evaluate levels of the hSLC7A11 of the invention in tissue or bodily fluid.

Although the invention has been described in some detail by way of illustration and example for purposes of clarity and understanding, it will be apparent that certain changes and modifications may be practiced within the scope of the appended claims.

What is claimed is:

1. An isolated polynucleotide comprising:
(a) a nucleotide sequence encoding a solute carrier family 7, member 11 polypeptide wherein the amino acid sequence of the polypeptide and the amino acid sequence of SEQ ID NO: 4 have at least 80% sequence identity; or
(b) the complement of the nucleotide sequence, wherein the complement and the nucleotide sequence contain the same number of nucleotides and are 100% complementary.

2. The polynucleotide of claim 1 wherein the sequence identity is at least 98%.

3. The polynucleotide of claim I wherein the polynucleotide encodes the polypeptide of SEQ ID NO: 4.

4. The polynucleotide of claim 1 that comprises the nucleotide sequence of SEQ ID NO: 3.

5. An isolated polynucleotide comprising:
(a) a nucleotide sequence encoding a solute carrier family 7, member 11 polypeptide wherein the amino acid sequence of the polypeptide and the amino acid sequence of SEQ ID NO: 6 have at least 80% sequence identity; or
(b) the complement of the nucleotide sequence, wherein the complement and the nucleotide sequence contain the same number of nucleotides and are 100% complementary.

6. The polynucleotide of claim 5 wherein the sequence identity is at least 97%.

7. The polynucleotide of claim 5 wherein the polynucleotide encodes the polypeptide of SEQ ID NO: 6.

8. The polynucleotide of claim 5 that comprises the nucleotide sequence of SEQ ID NO: 5.

9. An expression vector comprising the polynucleotide of claim 1 and an expression control sequence operatively linked to the polynucleotide.

10. A process for producing a recombinant host cell comprising transforming or transfecting a host cell with the expression vector of claim 9 such that the host cell, under appropriate culture conditions, produces a solute carrier family 7, member 11 polypeptide.

11. A recombinant host cell produced by the process of claim 10.

12. An isolated solute carrier family 7, member 11 polypeptide comprising an amino acid sequence that has at least 80% sequence identity to the amino acid sequence of SEQ ID NO: 4.

13. The polypeptide of claim 12 wherein the sequence identity is at least 98%.

14. The polypeptide of claim 12 that comprises the amino acid sequence of SEQ ID NO: 4.

15. An isolated solute carrier family 7, member 11 polypeptide comprising an amino acid sequence that has at least 80% sequence identity to the amino acid sequence of SEQ ID NO: 6.

16. The polypeptide of claim 15 wherein the sequence identity is at least 97%.

17. The polypeptide of claim 15 that comprises the amino acid sequence of SEQ ID NO: 6.

18. A process for producing a solute carrier family 7, member 11 polypeptide comprising culturing the recombinant host cell of claim 11 under conditions sufficient for the production of said polypeptide and recovering the polypeptide from the culture.

19. A method for identifying a receptor which is capable of binding to a solute carrier family 7, member 11 molecule or a fragment thereof, said method comprising the steps of:
(a) reacting the solute carrier family 7, member 11 polypeptide of claim 12 or a fragment thereof with a candidate receptor under conditions which permit the formation of receptor-solute carrier family 7, member 11 polypeptide complexes; and
(b) assaying for candidate receptor-solute carrier family 7, member 11 polypeptide complexes or for activation of the candidate receptor, wherein the presence of at least one of candidate receptor-solute carrier family 7, member 11 polypeptide complexes and activation of the candidate receptor indicates that the candidate receptor is capable of binding to said solute carrier family 7, member 11 molecule or said fragment thereof.

20. A method for identifying a receptor which is capable of binding to a solute carrier family 7, member 11 molecule or a fragment thereof, said method comprising the steps of:
(a) reacting the solute carrier family 7, member 11 polypeptide of claim 15 or a fragment thereof with a candidate receptor under conditions which permit the formation of receptor-solute carrier family 7, member 11 polypeptide complexes; and
(b) assaying for candidate receptor-solute carrier family 7, member 11 polypeptide complexes or for activation of the candidate receptor, wherein the presence of at least one of candidate receptor-solute carrier family 7, member 11 polypeptide complexes and activation of the candidate receptor indicates that the candidate receptor is capable of binding to said solute carrier family 7, member 11 molecule or said fragment thereof.
